(19) United States
(12) Patent Application Publication
(10) Pub. No.: US 2026/0072026 A1
(43) Pub. Date: Mar. 12, 2026

(54) SOLID-PHASE CARRIER FOR DETECTING OR SEPARATING/PURIFYING CELLS AND/OR EXTRACELLULAR VESICLES

(71) Applicant: RNAi Inc., Tokyo (JP)

(72) Inventor: Takuya TAMATANI, Tokyo (JP)

(73) Assignee: RNAi Inc., Tokyo (JP)

(21) Appl. No.: 18/953,431

(22) Filed: Nov. 20, 2024

(30) Foreign Application Priority Data
Nov. 21, 2023 (JP) 2023-197552

(51) Int. Cl. G01N 33/552; G01N 1/30; G01N 33/569

(52) U.S. Cl. CPC G01N 33/552; G01N 1/30; G01N 33/569

(57) ABSTRACT

The purpose of the present invention is to provide a new technique for trapping cells and/or extracellular vesicles (EVs) without using antibodies. The present invention is a solid-phase carrier having an ability to adsorb cells and/or extracellular vesicles, and comprising a substrate and an ion exchanger.

### TECHNICAL FIELD

The present invention relates to a solid-phase carrier for detecting or separating/purifying cells and/or extracellular vesicles.

### BACKGROUND ART

Extracellular vesicles (EVs) are defined as particles which are released from cells, do not have nuclei (cannot be replicated), and are enclosed by a lipid bilayer membrane. Recent studies have revealed that almost all cells including those of animals such as humans as well as plants, fungi and bacteria release extracellular vesicles.

It is known that extracellular vesicles (EVs) contain informational substances (nucleic acids (DNA, mRNA, miRNA), proteins and lipids, and the like), and play an important role in intercellular signaling. It has been shown that not only extracellular vesicles released from tissue cells of organs in the body, but also EVs derived from intestinal bacteria and EVs derived from food and the like, which are taken up into the body, are delivered to distant organs through a bloodstream and function within the body.

Recently, it has come to be considered that main active ingredients contributing to the effects of functional foods, fermented foods, live biotherapeutic product (LBP) and the like are EVs. In addition, it has been revealed that the effects of cells for use in treatment, such as mesenchymal stem cells (MSCs) can also be replaced by the EVs they released. The advancement of studies on EVs has enhanced opportunities for EV drug development, and in the United States, more than 100 EV-based therapeutics are in clinical trial stages. It has come to be considered that EVs have a high potential as a target in future product development and drug development.

Meanwhile, in development of an EV-related product, it is necessary to detect and quantitate EVs, but it is difficult to analyze EVs that are very small in size, and the approach of analyzing EVs is currently limited to, for example, those that require massive equipment such as an electron microscope, or analysis with a flow cytometer which requires a complicated process such as attachment of beads to EVs (Non Patent Document 1). An approach of analyzing EVs easily and conveniently is desired.

Here, for analyzing EVs, it may be first necessary to develop a technique for trapping EVs. The method that has been heretofore well known is a method in which EVs are trapped by an antibody targeting a protein or the like on the surface of EVs. However, this method, in which an antibody is used, thus has a problem in terms of cost and inconvenient handling. In addition, there is a problem that EVs which can be trapped are limited to EVs which can be recognized by the relevant antibodies.

A technique for trapping cells is desired as well as a technique for trapping EVs.

### PRIOR ART DOCUMENTS

### Non Patent Document

Non Patent Document 1: J Extracell Vesicles. 2023 Feb.; 12 (2): e12299. doi: 10. 1002/jev2. 12299.

### SUMMARY OF INVENTION

### Technical Problem

In view of the circumstances described above, an object of the present invention is to provide a new technique for trapping cells and/or extracellular vesicles (EVs) without using antibodies.

### Solution to Problem

The present inventor has conducted intensive studies for the above-described object, and resultantly found that surprisingly, an ion exchanger can effectively trap extracellular vesicles (EVs), leading to completion of the present invention. Specifically, the present invention provides the following.
[1] A solid-phase carrier having an ability to adsorb cells and/or extracellular vesicles, the solid-phase carrier comprising a substrate and an ion exchanger.

[2] The solid-phase carrier according to [1], wherein the ion exchanger and the substrate are bound to each other by a covalent bond, either via a connection part or without a connection part.

[3] The solid-phase carrier according to [1] or [2], wherein the ion exchanger is a weak anion exchanger having a secondary and/or tertiary amino group as a partial structure.
[4] The solid-phase carrier according to [3], wherein the ion exchanger is a monovalent residue or a polyvalent residue derived from polyethyleneimine.
[5] The solid-phase carrier according to [4], wherein the polyethyleneimine is branched high-molecular-weight polyethyleneimine.
[6] The solid-phase carrier according to any one of [1] to [5], wherein the ion exchanger and the substrate are bound to each other via a connection part, and the connection part comprises a silicon-carbon bond.
[7] The solid-phase carrier according to any one of [1] to [6], wherein a material of the substrate is glass or metal oxide.
[8] The solid-phase carrier according to any one of [1] to [7], wherein the substrate is a glass slide or a microscope glass plate.
[9] The solid-phase carrier according to [8], wherein a material of the glass slide or the microscope glass plate is glass having a transmittance of 30% or more at a wavelength of 300 nm.
[10] The solid-phase carrier according to [8] or [9], wherein in the glass slide or the microscope glass plate, a surface roughness (Ra) of the glass before the ion exchanger is fixed thereon is 10 nm or less.
[11] The solid-phase carrier according to any one of [1] to [10], wherein a surface of the solid-phase carrier is two-dimensionally separated into a hydrophilic region and a water-repellent region.
[12] The solid-phase carrier according to any one of [1] to [7], wherein the substrate is a column filler.
[13] The solid-phase carrier according to any one of [1] to [7], wherein the substrate is a plate for a separation/purification stack.
[14] A kit for detecting or quantitating cells and/or extracellular vesicles, comprising the solid-phase carrier according to any one of [1] to [13], and a substance for detecting cells and/or extracellular vesicles.
[15] The kit according to [14], for use in examination/diagnosis.
[16] The kit according to [15], wherein a target for the examination/diagnosis is at least one selected from the group consisting of a bowel disease, a liver disease, a kidney disease, a neuropsychiatric disease, a metabolic disease, a respiratory disease, a cardiovascular disease, an age-related disease, and heavy alcohol intake.
[17] A method for detecting cells and/or extracellular vesicles, comprising: adsorbing cells and/or extracellular vesicles onto the solid-phase carrier according to any one of [1] to [13]; and detecting the cells and/or extracellular vesicles adsorbed onto the solid-phase carrier.
[18] The detection method according to [17], further comprising: labeling cells and/or extracellular vesicles with a substance for detecting cells and/or extracellular vesicles.
[19] The detection method according to [18], wherein the substance for detecting cells and/or extracellular vesicles is at least one selected from the group consisting of an antibody, lectin, a membrane-reactive substance, and a nucleic acid-reactive substance.
[20] The detection method according to any one of [17] to [19], wherein cells and/or extracellular vesicles are detected by microscope observation.
[21] The detection method according to any one of [17] to [19], wherein cells and/or extracellular vesicles are detected by an immunoassay method.
[22] The detection method according to any one of [17] to [21], for use in examination/diagnosis.
[23] The detection method according to [22], wherein a target for the examination/diagnosis is at least one selected from the group consisting of a bowel disease, a liver disease, a kidney disease, a neuropsychiatric disease, a metabolic disease, a respiratory disease, a cardiovascular disease, an age-related disease, and heavy alcohol intake.
[24] A column for separating/purifying cells and/or extracellular vesicles, filled with the solid-phase carrier according to [12].
[25] A stack for separating/purifying cells and/or extracellular vesicles, comprising the solid-phase carriers according to [13] stacked as a stack plate.
[26] A method for separating/purifying cells and/or extracellular vesicles, comprising: bringing a solution containing cells and/or extracellular vesicles of interest into contact with the solid-phase carrier according to any one of [1] to [13]; and eluting cells and/or extracellular vesicles of interest adsorbed onto the solid-phase carrier with an eluate.
[27] A method for separating/purifying cells and/or extracellular vesicles, comprising: bringing a solution containing cells and/or extracellular vesicles of interest into contact with the solid-phase carrier according to any one of [1] to [13]; and collecting cells and/or extracellular vesicles of interest not adsorbed onto the solid-phase carrier.
[28] A pharmaceutical composition comprising cells and/or extracellular vesicles separated/purified by the separation/purification method according to [26] or [27].
[29] A method for fixing cells and/or extracellular vesicles onto a solid-phase carrier, comprising: adsorbing cells and/or extracellular vesicles onto the solid-phase carrier according to any one of [1] to [13].

Two or more of the constitutions of [1] to [29] can be arbitrarily selected and combined.

### Advantageous Effects of Invention

According to the present invention, a new technique for trapping extracellular vesicles (EVs) can be provided. The technique of the present invention, in which not an antibody but an ion exchanger is used for trapping EVs, thus is advantageous in terms of cost and ease of handling. In the technique of the present invention, cells can also be targeted for trapping.

### BRIEF DESCRIPTION OF DRAWINGS

FIG. 1 shows photographs demonstrating that Escherichia coli-derived EVs, lactic acid bacteria-derived EVs or lemon-derived EVs can be fixed onto a PEI-fixed glass slide, and detected with a fluorescent microscope.

FIG. 2 shows photographs demonstrating that human cell culture supernatant-derived EVs or EVs in a human urine sample can be fixed onto a DEAE-fixed glass slide, and detected with a fluorescent microscope.

FIG. 3 shows photographs demonstrating that Gram-positive bacteria-derived EVs and Gram-negative bacteria-derived EVs can be detected at the same time on a PEI-fixed glass slide with a fluorescent microscope.

FIG. 4 shows photographs demonstrating that EVs in a human blood plasma sample can be fixed onto a PEI-fixed glass slide, and detected with a super-resolution microscope.

### DESCRIPTION OF EMBODIMENTS

Hereinafter, the present invention will be described in detail, but the present invention is not limited thereto. The following descriptions of the solid-phase carrier, the kit, the detection method, the separation/purification method, the examination/diagnosis method, the fixation method and the like of the present invention can be mutually referenced and applied.

### Solid-Phase Carrier

A solid-phase carrier of the present invention comprises a substrate and an ion exchanger. The solid-phase carrier of the present invention, which comprises an ion exchanger, thus has an ability to adsorb cells and/or extracellular vesicles.

In the present invention, the ion exchanger is, for example, directly or indirectly bound and fixed to the substrate at a part or the whole of a surface of the substrate.

Here, the mode of binding of the substrate and the ion exchanger is not limited, and the substrate and the ion exchanger may be bound to each other by a covalent bond, or by an attracting interaction other than a covalent bond. The ion exchanger can be directly bound by a modification reaction of the substrate, or the ion exchanger can be indirectly bound to the substrate by a multistep modification reaction. In the present invention, the mode of binding is preferably the binding by a covalent bond.

When the ion exchanger is indirectly bound onto the substrate by a multistep modification reaction, a modification part which is other than the substrate and the ion exchanger, and present therebetween is referred to as a connection part. Here, it can be said that the ion exchanger is indirectly bound to the surface of the substrate via the connection part. The connection part can also be called, for example, a linker or a spacer. For example, a silane coupling agent having a functional group for introduction of an ion exchanger at the end (for example, 3-hydroxypropyl triethoxysilane or 3-aminopropyl triethoxysilane) can connect the functional group to the surface of glass (substrate) by a covalent bond. By reacting diethylaminoethyl chloride hydrochloride with the surface functional group introduced as described above, a glass surface to which diethylaminoethyl is bound is obtained. A glass surface to which diethylaminoethyl is bound is also obtained by reacting glutaraldehyde as a cross-linker and 2-diethylaminoethylamine with the surface functional group. In the present invention, here, the moiety of diethylaminoethyl (DEAE) is referred to as an ion exchanger, and the moiety closer to the glass is referred to as a connection part.

### Ion Exchanger

In the present invention, the term “ion exchanger” is used in a commonly used sense in the relevant technical field. Specifically, the term refers to a cation or a partial structure having an attracting interaction with an anion in a so-called ion-exchange substance such as an ion-exchange resin. The ion exchanger is classified into a strong anion exchanger, a weak anion exchanger, a strong cation exchanger and a weak cation exchanger. As the ion exchanger in the present invention, a strong anion exchanger and a weak anion exchanger can be preferably used, and a weak anion exchanger can be more preferably used.

In the present invention, the strong anion exchanger means an ion exchanger that retains a cationic property even at a high pH, which has a partial structure such as quaternary ammonium, N,N-dialkylimidazolium or N-alkylpyridinium. Specific examples thereof include partial structures such as tetraalkylammonium (for example, trimethylammonioethyl, trimethylammoniopropyl, triethylammonioethyl or triethylammoniopropyl), 1-alkylpyridinium (for example,1-methylpyridinium-4-yl or 1-ethylpyridinium-3-yl), and 1,3-dialkylimidazolium (for example, 1,3-dimethylimidazolium-4-yl).

In the present invention, the weak anion exchanger means an ion exchanger that loses a cationic property at a high pH, which has a partial structure as a monovalent residue or a polyvalent residue derived from primary to tertiary amine, imidazole, monoalkylimidazole, N-unsubstituted pyrimidine or the like. Specific examples thereof include partial structures such as a secondary amino group (for example, methylaminoethyl, ethylaminoethyl or methylaminopropyl), a tertiary amino group (for example, dimethylaminoethyl, dimethylaminopropyl, diethylaminoethyl, diethylaminopropyl, a pyridyl group, a 1-methylimidazolyl group or a 1-ethylimidazolyl group), and partial structures as a monovalent residue or a polyvalent residue derived from polyethyleneimine. In the present invention, diethylaminoethyl (DEAE), and a monovalent residue or a polyvalent residue derived from polyethyleneimine can be preferably used. In the present specification, polyethyleneimine, and a monovalent residue or a polyvalent residue derived from polyethyleneimine may be referred to as PEI.

In the present invention, any type of polyethyleneimine can be used. For example, unbranched linear low-molecular-weight polyethyleneimine, branched low-molecular-weight polyethyleneimine, unbranched linear high-molecular-weight polyethyleneimine, and branched high-molecular-weight polyethylene imine can be used, and among them, branched high-molecular-weight polyethyleneimine can be preferably used. In the present invention, selectivity to cells and/or extracellular vesicles adsorbed can be varied depending on the type of polyethyleneimine, which can be appropriately selected for use according to the purpose.

In the present invention, the strong cation exchanger means an ion exchanger that retains an anionic property even at a low pH, which has a partial structure such as a strongly acidic group (partial structure) such as a sulfonic acid group (sulfo group) or a phosphonic acid group. Specific examples thereof include partial structures such as a sulfophenyl group, and a sulfoalkyl group (for example, a sulfomethyl group, a sulfoethyl group or a sulfopropyl group).

In the present invention, the weak cation exchanger means an ion exchanger that loses an anionic property at a low pH, which has a weakly acidic group (partial structure) such as carboxylic acid group (carboxyl group), or the like. Specific examples thereof include a carboxyalkyl group (for example, carboxymethyl or carboxyethyl).

### Substrate

In the present invention, the substrate means a substance capable of holding an ion exchanger. As described above, the ion exchanger is directly or indirectly bound and fixed to a part or the whole of a surface of the substrate. The substrate that can be used in the present invention is not limited, and examples thereof include substrates such as devices, tools, materials and reagents which are commonly used in the field of biological detection methods or quantitation methods, or separation/purification methods. More specific examples of the substrate include a glass slide, a microscope glass plate, a cover glass, a well plate, a microplate, a dish, a flask, a column filler, a plate for a separation stack, and a separating bead (for example, a magnetic bead).

In the present invention, the material of the substrate is not limited. Materials of substrates (including devices, tools, materials, reagents and the like) which are commonly used in the field of biological detection methods or quantitation methods, or separation/purification methods can be used. Examples of the material of the substrate include glass, plastic, metals, resins, polysaccharides, paper, and cloth. In the present invention, glass, and metal oxide can be preferably used.

In the present invention, the shape of the substrate is not limited, and may be the shape of any of substrates (including devices, tools, materials, reagents and the like) which are commonly used in the field of biological detection methods or quantitation methods, or separation/purification methods. Examples of the shape of the substrate include a particle shape, a filament shape, a net shape, a porous shape, a fiber shape, a film shape, a plate shape, a dish shape, and a cylinder shape.

### Cells

In the present invention, the term “cell” is used in a commonly used sense in the relevant technical field. In the present invention, the cells to be targeted may be arbitrary cells (for example, cells), and for example, cells of animals (including humans), plant cells, and microorganism cells (including bacteria, fungi and the like) can be targeted. In the present invention, floating cells may be targeted, or adhered cells may be targeted, and floating cells and adhered cells can be targeted.

### Extracellular Vesicles

In the present invention, the term “extracellular vesicle (EV)” is used in a commonly used sense in the relevant technical field, referring to, for example, particle which is released from cells, does not have nuclei (cannot be replicated), and is enclosed by a lipid bilayer membrane. The extracellular vesicles are classified mainly into exosomes, microvesicles and apoptotic bodies. The exosome is a vesicle having a size of about 50 to 200 nm and derived from an endosome membrane, being formed in an endocytosis process, which is composed mainly of lipids, proteins and nucleic acids. The microvesicle is a vesicle whose size is in a broad range of 100 to 1,000 nm. The microvesicle is different from the exosome in that it is generated directly from the cell membrane and secreted to the outside of the cell, but it is difficult to completely distinguish the microvesicle from the exosome. The apoptotic bodies is a particle generated from the membrane in a cell having undergone apoptosis, which has a size of micrometer order. The extracellular vesicle has lipids, proteins and the like derived from the cell membrane of a parent cell on the surface thereof, contains nucleic acids such as mRNA and miRNA, and proteins, and includes information derived from the parent cell. In the present invention, the extracellular vesicles to be targeted may be extracellular vesicles derived from arbitrary living cells. For example, extracellular vesicles derived from animals (including humans), plants, microorganisms (including bacteria, fungi and the like), and the like can be targeted.

### Adsorption Ability

In the solid-phase carrier of the present invention, the ion exchanger has an action of adsorbing cells and/or extracellular vesicles, and an ability to adsorb cells and/or extracellular vesicles onto the solid-phase carrier is imparted to the solid-phase carrier by the ion exchanger. In the present invention, the solid-phase carrier comprising an ion exchanger has an ability to adsorb cells and/or extracellular vesicles, and for example, may be said to have an ability to adsorb cells, said to have an ability to adsorb extracellular vesicles, or said to have an ability to adsorb cells and extracellular vesicles. For example, when the solid-phase carrier is said to have an ability to adsorb extracellular vesicles, it is only required to have an ability to adsorb extracellular vesicles, and it may be or may not be required to have an ability to adsorb cells.

Here, the ability of the ion exchanger to adsorb various extracellular vesicles varies depending on the type thereof, and a type of extracellular vesicles capable of being adsorbed by the solid-phase carrier can be selected by selecting an ion exchanger according to a purpose. For example, when diethylaminoethyl (DEAE) is used as the ion exchanger, the solid-phase carrier can adsorb arbitrary extracellular vesicles without regard to, for example, an origin of the extracellular vesicles (for example, humans, plants or bacteria). On the other hand, when a monovalent residue or a polyvalent residue derived from polyethyleneimine is used as the ion exchanger, the solid-phase carrier can adsorb extracellular vesicles derived from plants and bacteria, but hardly adsorbs extracellular vesicles derived from humans. Similarly, the ability of the ion exchanger to adsorb cells varies depending on the type thereof, and a type of cells capable of being adsorbed by the solid-phase carrier can be selected by selecting an ion exchanger according to a purpose.

### Preferred Aspects

### (Glass Slide, Microscope Glass Plate and Cover Glass)

When the solid-phase carrier of the present invention is used for detection and quantitation of cells and/or extracellular vesicles, it is effective to use various optical microscopes (including fluorescent microscopes) (note that the optical microscopes includes various super-resolution microscopes). Here, a solid-phase carrier, in which a substrate for use in fixation of cells and/or extracellular vesicles to be observed in the observation with various optical microscopes (specifically, a glass slide, a microscope glass plate, a cover glass or the like) is used, and an ion exchanger is fixed on a surface of the substrate, can be preferably used. In the present invention, since the solid-phase carrier is substantially a substrate on which an ion exchanger is fixed, the solid-phase carrier may be called by the name of the substrate (for example, a solid-phase carrier in which a glass slide is used as a substrate, and an ion exchanger is fixed on a surface thereof may be called a glass slide).

The outline of the above detection/quantitation method using various optical microscopes (including fluorescent microscopes) is as follows. First, a liquid sample which is a specimen (blood, blood plasma, serum, urine, fruit juice, a culture solution of fungi or bacteria, or the like) is placed on the surface of the solid-phase carrier having a glass slide, a microscope glass plate or a cover glass as a substrate, and left standing for a certain time to adsorb cells and/or extracellular vesicles onto the surface of the solid-phase carrier. Next, only a target to be detected is labeled (for example, fluorescent staining) using a reagent capable of labeling (including various staining reagents). Finally, the solid-phase carrier is thoroughly washed to remove an unreacted reagent, and the labeled target is observed with an optical microscope (including a fluorescent microscope) to perform the detection. Depending on a purpose, it is also possible to perform quantitation by counting the number of labeled targets.

When a target is observed (detected) with an optical microscope, the observation surface is preferably as smooth as possible. The studies of the present inventor have showed that if the observation surface (a surface onto which cells and/or extracellular vesicles are adsorbed) is not smooth, EVs which are very small particles cannot uniformly adsorb onto the observation surface, and subsequent observation (detection) is likely to be difficult. Therefore, the observation surface should be as smooth as possible. Such an observation surface is given by a glass slide, a microscope glass plate or a cover glass, in particular, a glass slide, a microscope glass plate or a cover glass using glass having a small surface roughness (Ra value). The surface roughness of the substrate for use in the present invention is preferably 20 nm or less, more preferably 10 nm or less, and particularly preferably 5 nm or less. In the present specification, the “surface roughness (Ra)” is an arithmetic average roughness determined according to JIS B 0601 (2013).

When the target is observed (detected) using a fluorescent microscope, background fluorescence generated by the substrate may sometimes become a problem. For preventing this, it is preferable to use, rather than mere soda-lime glass (blue plate glass or water plate glass), glass obtained by mixing silica with soda-lime glass to suppress generation of fluorescence (for example, white plate glass, half-white glass or fine glass when exemplified on the basis of the product classification of Matsunami Glass Ind., Ltd.). Glass suppressing generation of fluorescence is characterized by having a high transmittance at a wavelength of 300 nm. Thus, in the glass used as a substrate in the present invention, the transmittance at this wavelength is preferably 30% or more, more preferably 40% or more, and particularly preferably 50% or more.

When the target is observed (detected) using the solid-phase carrier of the present invention, and the number of the targets is counted to perform quantitation, it is necessary to exactly know the liquid amount of a liquid sample placed on the solid-phase carrier (specifically, a glass slide, a microscope glass plate, a cover glass or the like) and the spread area of the liquid sample spread on the substrate. The former can be known by using a high-precision quantitative pipette, and the latter can be known by defining a wet spread of a liquid sample (in many cases, a liquid sample containing water as a main component). That is, a specific region of a sample-supported surface of the solid-phase carrier (specifically, a glass slide, a microscope glass plate, a cover glass or the like) is made water-repellent to control the wet spread of the liquid sample (a water-based liquid), thereby defining the spread area of the liquid sample. In the present invention, for example, it is preferable that the surface of the solid-phase carrier be two-dimensionally separated into a hydrophilic region and a water-repellent region to define the spread area of the liquid sample on the surface of the solid-phase carrier.

The method for separation between a hydrophilic region and a water-repellent region is not limited. For example, the separation may be performed by printing a specific two-dimensional shape (typically a shape in which the hydrophilic region forms a circle) on a hydrophilic surface with water-repellent ink, or a ready-made product suitable as the substrate in the present invention may be used if available. The separation between a hydrophilic region and a water-repellent region may be firstly performed on the substrate before fixation of the ion exchanger, or may be performed at any stages, for example, after introduction of a connection part, after fixation of the ion exchanger, or the like. Ready-made products are commercially available in which a connection part having a functional group is introduced into a substrate (for example, a glass slide, a microscope glass plate or a cover glass), the upper part of which is separated into a hydrophilic region and a water-repellent region. It is particularly convenient to use such a commercial product when a solid-phase carrier for quantitative measurement is prepared. In the case where such a commercial product is used, the ion exchanger is fixed by subsequent chemical reaction treatment to only a substrate surface where water-repellent ink is not applied. Here, the method for fixing the ion exchanger is not limited, and examples thereof include a method in which an aqueous solution in which the ion exchanger is dissolved is added dropwise to the hydrophilic region and reacted with the functional group of the connection part. Examples of the typical reaction include a reaction of a hydroxyl group or an amino group of the connection part and diethylaminoethyl chloride hydrochloride as described above.

For example, when a polyethyleneimine residue which is a preferred ion exchanger for the present invention is connected to a connection part having an amino group, a difunctional electrophile is used as a cross-linker. Examples of the preferred difunctional electrophile include dialdehydes (glutaraldehyde, succinaldehyde, and the like). Of these, glutaraldehyde is preferably used because of high water solubility.

In the example described above, there is only a little level difference between a region printed with a water-repellent agent (water-repellent ink) and a region which is not printed with the agent, but when an appropriate amount of an aqueous solution is added dropwise to the hydrophilic region, the aqueous solution is held in a hemispheric shape in the hydrophilic region, and does not leak out to the water-repellent region. Since the hydrophilic region functions as if it were a well (hole) that holds an aqueous solution, the hydrophilic region may be referred to as a “hole”. Therefore, a glass slide having 12 hydrophilic regions defined by printing of a water-repellent agent may be referred to as a 12-hole glass slide.

### (Column Filler)

When the solid-phase carrier of the present invention is used for separating/purifying cells and/or extracellular vesicles, it is effective to use the solid-phase carrier in various columns. Here, a solid-phase carrier, in which a common column filler is used as a substrate, and an ion exchanger is fixed on a surface of the substrate, is preferably used.

In the present invention, the column filler that can be used is not limited as long as it is a column filler which is commonly used in the field of biological separation/purification methods. The material of the column filler may be an inorganic or organic material. Examples of the inorganic material include silicon, titanium, zinc, and aluminum, and examples of the organic material include polysaccharides such as agarose, cellulose, chitin, chitosan, amylose, heparin, hyaluronic acid and pectin, polyacrylamide, polyacrylic acid, polystyrene, polyvinyl alcohol, polymethacrylic acid, polyacrylic acid, polymethacrylic acid esters, polyacrylic acid esters, and derivatives thereof. The shape of the column filler may be, for example, a shape like particles, more specifically, a shape like porous particles or non-porous particles, but is not limited thereto.

An aspect of the present invention is a column for separating/purifying cells and/or extracellular vesicles, filled with the solid-phase carrier of the present invention having a column filler as a substrate. Specifically, a separation/purification column, in which the inside of a column is filled with the solid-phase carrier of the present invention having a column filler as a substrate as described above, is within the scope of the present invention.

### Plate for Separation/Purification Stack

When the solid-phase carrier of the present invention is used for separating/purifying cells and/or extracellular vesicles, the solid-phase carrier may be, for example, a stack plate of a separation/purification stack. In the solid-phase carrier as a stack plate, the substrate thereof is only required to be a substrate that can be used as a stack plate (a plate for a stack). Normally, the shape of the plate for a stack is a plate shape, and the material thereof is not limited. Therefore, a stack plate (solid-phase carrier) obtained by fixing an ion exchanger on a surface of a plate for a stack (substrate) can be widely used. However, examples of the preferred material of the substrate include metals, and ceramic.

In the present invention, the separation/purification stack means a type of device or apparatus for separating/purifying cells and/or extracellular vesicles, in which plate-shaped materials (stack plates) having an ability to adsorb cells and/or extracellular vesicles are stacked and placed inside a box-shaped container, and an unseparated/unpurified liquid sample (for example, a specimen) flows into the box-shaped container, where a target is adsorbed onto the stack plate to separate/purify cells and/or extracellular vesicles. Normally, the box-shaped container includes an inlet for allowing the liquid sample to flow into the container from the outside, and an outlet for allowing the liquid sample to flow from the inside of the container to the outside, so that the liquid sample passes through the inside of the container of the separation/purification stack, whereby a target can be separated/purified in the same manner as with a column. For the box-shaped container of the separation/purification stack, an open container having no lid portion in the upper part of the box, or a closed-type container having a lid portion in the upper part of the box can be used, and any of these types of containers can be selected according to a purpose.

An aspect of the present invention is a stack for separating/purifying cells and/or extracellular vesicles in which the solid-phase carriers of the present invention are stacked and provided as stack plates. Specifically, a separation/purification stack, in which the solid-phase carriers of the present invention having a plate for a stack as a substrate (that is, stack plates) are stacked and provided as described above, is within the scope of the present invention.

### Kit

A kit of the present invention comprises the solid-phase carrier of the present invention, and a substance for detecting cells and/or extracellular vesicles, and is used for detecting or quantitating cells and/or extracellular vesicles.

The kit of the present invention comprises the solid-phase carrier of the present invention, and a substance for detecting cells and/or extracellular vesicles, with no limitation on other constituents. The kit may include arbitrary reagents, devices and the like for use in detection or quantitation, and may include written instructions for use of the present kit which enable appropriate use of the contents of the kit of the present invention in detection or quantitation. The kit of the present invention can be used for a detection method or a quantitation method described below.

The kit of the present invention may be, for example, a kit for use in examination/diagnosis. The kit of the present invention can be used for the detection method or the quantitation method or an examination/diagnosis method of the present invention which will be described below.

### Detection Method

A method for detecting cells and/or extracellular vesicles according to the present invention comprises: adsorbing cells and/or extracellular vesicles onto the solid-phase carrier of the present invention; and detecting the cells and/or extracellular vesicles adsorbed onto the solid-phase carrier, with no limitation on other steps.

### (Adsorption Step)

The adsorption step in the detection method of the present invention is a step of adsorbing cells and/or extracellular vesicles onto the solid-phase carrier of the present invention. By this step, cells and/or extracellular vesicles to be detected can be adsorbed onto the solid-phase carrier, and thus fixed onto the solid phase, so that the cells and/or extracellular vesicles are easily detected. Here, cells and/or extracellular vesicles adsorbed onto the solid-phase carrier are typically provided as a solution containing the cells and/or extracellular vesicles (also referred to as a liquid sample), and the liquid sample is brought into contact with the solid-phase carrier of the present invention to adsorb cells and/or extracellular vesicles of interest onto the solid-phase carrier.

The liquid sample in the present invention may be, for example, a solution containing purified (or partially purified) cells and/or extracellular vesicles, or a solution containing unpurified cells and/or extracellular vesicles. Examples of the unpurified liquid sample include blood, blood plasma, serum, urine, saliva, tears, cerebrospinal fluid, fruit juice, cell culture solutions, and culture solutions of fungi and bacteria.

In the present invention, the selectivity on cells and/or extracellular vesicles depends on the ion exchanger fixed on the solid-phase carrier. Thus, in the detection method of the present invention, the solid-phase carrier is selected for use according to cells and extracellular vesicles to be detected.

Here, in the case of use of a liquid sample, the solution of the liquid sample is adjusted so that it has an osmotic pressure equal to that of body fluid, for example. Such a liquid sample is favorable for cells and/extracellular vesicles to be adsorbed onto the solid-phase carrier when the liquid sample and the solid-phase carrier of the present invention come into contact with each other.

In the present invention, adsorption of cells and/or extracellular vesicles onto the solid-phase carrier, in the case of use of a liquid sample, is advantageous in that the adsorption is achieved only by, for example, bringing the liquid sample and the solid-phase carrier into contact with each other, and other complicated operations are not required. More specifically, for example, in the case of a solid-phase carrier whose substrate is any of various detection plates such as a glass slide, a microscope glass plate, a cover glass, a well plate and a microplate, or a cell culture container such as a dish or a flask, a liquid sample is placed on the surface of the solid-phase carrier, and left standing for a predetermined time to adsorb cells and/or extracellular vesicles onto the solid-phase carrier, so that the cells and/or extracellular vesicles are fixed onto the solid-phase carrier. Here, for example, the method of the present invention enables easy and convenient fixation without necessity application of a centrifugal force using a centrifuge or the like even when the target is cells or extracellular vesicles that are likely to float. The condition for the adsorption reaction is such that the temperature is in the range of 0° C. to 60° C. The temperature is preferably 1° C. to 37° C., and more preferably 2° C. to 25° C. The condition of the reaction time is such that the reaction is carried out by still standing for 30 minutes or more, preferably 1 hour or more, and more preferably 2 hours or more.

Thereafter, the solid-phase carrier may be washed with a wash solution if necessary. For example, by washing the solid-phase carrier with a wash solution under conditions which ensure that the cells and/or extracellular vesicles are not detached from the solid-phase carrier, undesired impurities can be more effectively removed. As the wash solution, for example, a solution having little influence on cells and/or extracellular vesicles, such as PBS (phosphate buffered saline), can be preferably used. When immediately followed by detection by microscope observation, the washing is preferably performed using ion-exchange water, distilled water or the like as a wash solution for use in final washing. Washing with ion-exchange water, distilled water or the like enables prevention of precipitation of salts during microscope observation, which makes the observation difficult.

### (Labeling Step)

In detection of cells and/or extracellular vesicles, the cells and/or extracellular vesicles fixed on the solid-phase carrier can be directly observed (detected) by microscope observation or the like, but indirect detection is effective in which cells and/or extracellular vesicles to be detected are labeled with a substance for detecting cells and/or extracellular vesicles (hereinafter, also referred to as a detection substance), and the label is detected. Thus, the detection method of the present invention may further comprise the step of labeling cells and or extracellular vesicles with a substance for detecting cells and or extracellular vesicles (labeling step).

In the present invention, the detection substance can be used without limitation as long as it is a detection substance that is commonly used in the relevant technical field. The detection substance is appropriately selected normally according to a type of detection method, and a type of cells and/or extracellular vesicles to be detected, and can be appropriately selected without particular difficulty by those skilled in the relevant technical field. Specific examples of the detection substance include antibodies (for example, (FITC labeled) anti-CD9 antibody and (rhodamine labeled) anti-CD63 antibody), lectin, membrane-reactive substances (for example, (rhodamine labeled) polymyxin B, (FITC labeled) vancomycin and DiI dye), and nucleic acid-reactive substances (for example, SYTO). These detection substances can specifically label target cells and/or extracellular vesicles by, for example, recognizing a marker (a proteins, a sugar chain or the like) of the surfaces of cells and/or extracellular vesicles to be detected, and specifically binding to the marker. In the present invention, the detection substance can be used singly, or used in combination of two or more thereof.

To the detection substance, for example, a signal substance capable of generating a detectable signal is directly or indirectly bound. For example, the detection substance itself may generate a signal. Examples of the form of the detection substance to which a signal substance is directly bound include antibodies and nucleic acids to which a fluorescent dye is covalently bound. Examples of the form of the detection substance to which a signal substance is indirectly bound include antibodies captured by secondary antibodies to which a signal substance is covalently bound (in the present invention, the antibodies and the like are referred to as detection substances even before being captured by secondary antibodies). Thus, the detection substance can generate a signal. Here, examples of the signal substance include fluorescent substances and radioactive isotopes which are substances that themselves generate a signal. Examples of the signal substance also include enzymes which generate a signal by catalyzing a reaction of other substances (for example, alkali phosphatase, peroxidase, β-galactosidase and luciferase). Examples of the fluorescent substance include fluorescent dyes such as fluorescein isothiocyanate (FITC), rhodamine and Alexa Fluor (registered trademark), and fluorescent proteins such as GFP. Examples of the radioactive isotope include ¹²⁵I, ¹⁴C, and ³²P. Here, the method of labeling with the detection substance is known in the relevant technical field, and can be appropriately carried out under various conditions for the detection substance and the like by those skilled in the relevant technical field.

Thereafter, the solid-phase carrier may be washed with a wash solution if necessary. For example, by washing the solid-phase carrier with a wash solution under conditions which ensure that cells and/or extracellular vesicles are not detached from the solid-phase carrier, undesired impurities (for example, an unbound detection substance) can be more effectively removed. As the wash solution, for example, a solution having little influence on cells and/or extracellular vesicles, such as PBS (phosphate buffered saline), can be preferably used. When detection is then performed by microscope observation, the washing is preferably performed using ion-exchange water, distilled water or the like as a wash solution for use in final washing. Washing with ion-exchange water, distilled water or the like enables prevention of precipitation of salts during microscope observation, which makes the observation difficult.

### (Detection Method)

The detection step in the detection method of the present invention is a step of detecting cells and/or extracellular vesicles adsorbed onto the solid-phase carrier of the present invention. By this step, cells and/or extracellular vesicles to be detected, which are fixed on the solid-phase carrier, are practically detected. In the present invention, the detection of cells and/or extracellular vesicles includes detection of, for example, each of individual cells and/or extracellular vesicles by microscope observation or the like, and detection of, for example, an arbitrary measured amount that can reflect the number or amount of cells and/or extracellular vesicles contained in a predetermined amount of a liquid sample (for example, the amount of signals from the signal substances bound directly or indirectly to specific cells and/or extracellular vesicles), where all of what is generally called detection in the relevant technical field is intended.

In the present invention, cells and/or extracellular vesicles can be detected by a detection method that is commonly used in the relevant technical field. Specifically, for example, the detection can be performed by a method such as microscope observation or an immunoassay method. By microscope observation or the like, cells and/or extracellular vesicles to be detected, which are fixed on the solid-phase carrier, can be directly observed (detected). On the other hand, it is also possible to perform indirect detection in which cells and/or extracellular vesicles are labeled with a detection substance, and a signal generated from the detection substance (typically, a signal generated from a signal substance directly or indirectly bound to the detection substance) is detected by various methods. When the signal substance is, for example, an enzyme which generates a signal by catalyzing a reaction of other substances, the detection requires the use of a substrate of the enzyme, but such a detection method is known to those skilled in the relevant technical field, and those skilled in the relevant technical field can appropriately select a suitable enzyme according to a purpose, and use a substrate to detect a signal.

### Preferred Aspects

In the detection of the present invention, a solid-phase carrier suitable for d detecting cells and/or extracellular vesicles is used. For example, solid-phase carriers with substrates such as devices, tools, materials, reagents or the like that are commonly used in the field of biological detection methods can be preferably used. More specifically, a solid-phase carrier in which a glass slide, a microscope glass plate, a cover glass or the like is used as a substrate, and an ion exchanger is fixed on a surface of the substrate, can be preferably used. Such a solid-phase carrier is suitable for detection by microscope observation. For example, cells and/or extracellular vesicles are adsorbed and fixed onto a surface of the solid-phase carrier, the cells and/or extracellular vesicles can be labeled by fluorescent staining or the like on the intended surface, followed by observation (detection) of the labeled cells and/or extracellular vesicles with a fluorescent microscope.

In addition, a solid-phase carrier in which a detection plate such as a well plate or a microplate is used as a substrate, and an ion exchanger is fixed on a surface of the substrate, can be preferably used. Such a solid-phase carrier is suitable for detection by an immunoassay method. For example, cells and/or extracellular vesicles are adsorbed and fixed onto a surface of the solid-phase carrier, the cells and/or extracellular vesicles can be detected by an immunoassay method (for example, an ELISA method). At this time, a detection reagent containing a detection substance (for example, an enzyme-labeled antibody and a substrate of the enzyme) is appropriately selected for use according to an immunoassay method, and such a detection reagent can be appropriately selected by those skilled in the relevant technical field.

### Quantitation Method

The quantitation method of the present invention comprises: detecting cells and/or extracellular vesicles of interest by the detection method of the present invention; and quantitating the detected cells and/or extracellular vesicles, with no limitation on other steps.

### (Detection Step)

The detection step in the quantitation method of the present invention is a step of detecting cells and/or extracellular vesicles of interest by the above-described detection method of the present invention. By this step, cells and/or extracellular vesicles of interest can be detected. Here, for the detection step, the description of the above-described detection method of the present invention can be applied.

### (Quantitation Step)

The quantitation step in the quantitation method of the present invention is a step of quantitating cells and/or extracellular vesicles detected by the above-described detection step. For example, when individual cells and/or extracellular vesicles are each detected by microscope observation or the like, the quantitation can be performed by counting the number of the observed (detected) cells and/or extracellular vesicles. Here, when the detection is performed via a detection substance capable of specifically labeling specific cells and/or extracellular vesicles, the number of cells and/or extracellular vesicles of each type can be quantitated. This is advantageous in the case of microscope observation because the numbers of cells and/or extracellular vesicles of various types can be quantitated at one time. For example, when cells and/or extracellular vesicles are detected by an immunoassay method (for example, an ELISA method), cells and/or extracellular vesicles of various types can be quantitated by detection in which a specific antibody is bound to cells and/or extracellular vesicles of interest, and an enzyme reaction by the labeling enzyme bound directly or indirectly to the antibody is detected using an appropriate substrate. Quantitation by an immunoassay method is advantageous in that it can be carried out relatively easily and conveniently.

Here, as one example, an example will be shown in which extracellular vesicles fixed on the solid-phase carrier of the present invention (the substrate is a glass slide) are quantitated (quantitation step) with a fluorescent microscope. First, a fluorescent microscope photograph of extracellular vesicles fixed on the solid-phase carrier is acquired. The acquired fluorescent microscope photograph is analyzed with image analysis software capable of counting extracellular vesicles (cell-counting software or the like; for example, analysis application BZ-H4C from KEYENCE CORPORATION). Preferably, the image analysis software is, for example, capable of counting targets per specific area, capable of counting targets having a specified size, and capable of counting for each fluorescent dye in the case of a multi-stained sample. When such functions are available, the numbers of cells and extracellular vesicles can be separately counted, and the numbers of extracellular vesicles derived from different types of cells can be separately counted. If the amount of a liquid sample used for fixation on the solid-phase carrier is known, the concentration of extracellular vesicles of interest can be quantitated. For example, when a solid-phase carrier whose surface is two-dimensionally separated into a hydrophilic region and a water-repellent region is used, the accurate concentration of extracellular vesicles of interest can be easily quantitated.

Further, when a super-resolution microscope is used, specific molecules (for example, proteins and sugar chains) per one extracellular vesicle can also be counted, so that more detailed analysis can be made. The example here can be applied not only to extracellular vesicles, but also to cells.

As another example, an example will be shown in which extracellular vesicles fixed on the solid-phase carrier of the present invention (the substrate is a detection plate such as a well plate or a microplate) is quantitated (quantitation step) by an immunoassay method. First, extracellular vesicles are fixed on the solid-phase carrier. The solid-phase carrier is blocked with a blocking solution if necessary, and an antibody specific to extracellular vesicles of interest (for example, an antibody specific to a protein on the surfaces of extracellular vesicles of interest) is then brought into contact with the solid-phase carrier to label the extracellular vesicles. An excess antibody is removed with a wash solution, and if necessary (if the antibody is not labeled), an enzyme-labeled secondary antibody is then bound to the antibody. Thereafter, an excess antibody is removed with a wash solution, a substrate of the labeling enzyme is then brought into contact with the solid-phase carrier to induce an enzyme reaction, and a product of the enzyme reaction is detected and quantitated. In this way, the amount of extracellular vesicles of interest can be quantitated. The example here can be applied not only to extracellular vesicles, but also to cells.

### Examination/Diagnosis Method

The detection method or quantitation method of the present invention can be used for, for example, examination/diagnosis. In this case, the detection method or quantitation method of the present invention can also be referred to as, for example, an examination/diagnosis method.

In the present invention, the examination/diagnosis can be performed using, as an index, the amount of cells and/or extracellular vesicles detected or quantified by the detection method or quantitation method of the present invention, the amount of specific molecules (for example, proteins and sugar chains) per cell and/or extracellular vesicle, or the like. For example, by detecting or quantitating cancer cell-derived extracellular vesicles in a biological sample obtained from an examination/diagnosis subject (for example, a human subject), examination/diagnosis of various cancers can be made. For example, the detection method or quantitation method of the present invention is capable of detecting or quantitating the amount of lipopolysaccharide-positive bacteria extracellular vesicles in a biological sample obtained from an examination/diagnosis subject (by, for example, using fluorescently labeled polymyxin B, or an anti-lipopolysaccharide antibody), therefore, the amount of lipopolysaccharide-positive bacteria derived extracellular vesicles can be used as an index to make examination/diagnosis of a lipopolysaccharide-associated disease (and condition). For example, the detection method or quantitation method of the present invention is capable of detecting or quantitating the amount of amyloid β-containing extracellular vesicles in a biological sample obtained from an examination/diagnosis subject, therefore, the amount of amyloid-containing extracellular vesicles can be used as an index to make examination/diagnosis of Alzheimer's disease. For example, the detection method or quantitation method of the present invention is capable of detecting or quantitating the amount of extracellular vesicles containing an aging marker such GPNMB or GSL-1 in a biological sample obtained from an examination/diagnosis subject, therefore, the amount of extracellular vesicles containing an aging marker can be used as an index to make examination/diagnosis of the degree of procession of aging.

Here, diseases (and conditions) reported to involve a rise in blood lipopolysaccharide are known in the relevant technical field, and the examination/diagnosis of the present invention enables examination/diagnosis of such diseases (and conditions), for example. Specific examples of the disease (and condition) reported to involve a rise in blood lipopolysaccharide include the following.

Bowel diseases: ulcerative colitis/Crohn disease/irritable bowel syndrome; liver diseases: non-alcoholic fatty liver, alcoholic fatty liver, liver cirrhosis, hepatitis C, hepatitis B, liver cirrhosis, primary biliary cirrhosis, primary sclerosing cholangitis, liver fibrosis, liver cancer, and the like;

kidney diseases: chronic kidney disease, and the like;
neuropsychiatric disease: mild cognitive impairment (MCI), Parkinson's disease, Alzheimer's disease, multiple sclerosis, autism disorder, chronic fatigue syndrome, anxiety, depression, and the like;
metabolic diseases: obesity, type I diabetes, type II diabetes, potential diabetics, and the like; respiratory diseases: asthma, and the like;
cardiovascular diseases: ischemic heart disease, arterial sclerosis, angina pectoris, atrial fibrillation, pulmonary arterial hypertension, and the like, and;
other diseases and conditions: sepsis, rheumatoid arthritis, osteoarthritis, HIV, Covid-19, Graves'disease, ankylosing spondylitis, myalgic encephalomyelitis, heavy alcohol intake, sarcopenia, aging, and the like.

Therefore, the target for the examination/diagnosis of the present invention may, for example, any of the above-described diseases (and conditions), and preferably at least one selected from the group consisting of a bowel disease, a liver disease, a kidney disease, a neuropsychiatric disease, a metabolic disease, a respiratory disease, a cardiovascular disease, an age-related disease, and heavy alcohol intake.

It is known in the relevant technical field that patients suffering from an age-related disease has a higher blood lipopolysaccharide level as compared to healthy persons. Therefore, the target for the examination/diagnosis of the present invention may be, for example, an age-related disease. Among the diseases (and conditions) described above, non-alcoholic fatty liver, liver cirrhosis, liver fibrosis, liver cancer, chronic kidney disease, mild cognitive impairment (MCI), Parkinson's disease, Alzheimer's disease, obesity, type I diabetes, type II diabetes, potential diabetics, ischemic heart disease, arterial sclerosis, angina pectoris, atrial fibrillation, rheumatoid arthritis, osteoarthritis, sarcopenia and aging are age-related diseases.

### First Separation/Purification Method

The first method for separating/purifying cells and/or extracellular vesicles according to the present invention comprises: bringing a solution containing cells and/or extracellular vesicles of interest into contact with the solid-phase carrier of the present invention; and eluting cells and/or extracellular vesicles of interest adsorbed onto the solid-phase carrier with an eluate, with no limitation on other steps.

### (Contact Step)

The contact step in the first separation/purification method of the present invention is a step of bringing a solution containing cells and/or extracellular vesicles of interest (also referred to as a liquid sample) into contact with the solid-phase carrier of the present invention. In the first separation/purification method, by this step, the cells and/or extracellular vesicles of interest in the liquid sample are adsorbed onto the solid-phase carrier, and separated from undesired other impurities in the liquid sample.

In the present invention, as the liquid sample, an arbitrary solution containing cells and/or extracellular vesicles of interest can be used. For example, blood, blood plasma, serum, urine, saliva, tears, cerebrospinal fluid, fruit juice, a cell culture solution, a culture solution of fungi or bacteria, or the like can be used.

In the present invention, the selectivity on cells and/or extracellular vesicles depends on an ion exchanger fixed on the solid-phase carrier. Thus, in the first separation/purification method, the solid-phase carrier is selected for use according to cells and/or extracellular vesicles to be separated/purified.

Here, the solution of the liquid sample is adjusted so that it has an osmotic pressure equal to that of body fluid, for example. Such a liquid sample is favorable for cells and/extracellular vesicles to be adsorbed onto the solid-phase carrier when the liquid sample and the solid-phase carrier of the present invention come into contact with each other.

When the liquid sample and the solid-phase carrier of the present invention come into contact with each other, for example, the pH of an environment surrounding the anion-exchange solid-phase carrier is, for example, neutral or alkaline. Specifically, the pH may be, for example, 7 to 9.

After adsorption of cells and/or extracellular vesicles to the solid-phase carrier, undesired other impurities which have not adsorbed onto the solid-phase carrier are removed. For example, by washing the solid-phase carrier with a wash solution under conditions which ensure that cells and/or extracellular vesicles are not detached from the solid-phase carrier, undesired impurities can be more effectively removed. As the wash solution, for example, a solution having little influence on cells and/or extracellular vesicles, such as PBS (phosphate buffered saline), can be preferably used.

### (Elution Step)

The elution step in the first separation/purification method of the present invention is a step of eluting cells and/or extracellular vesicles of interest adsorbed onto the solid-phase carrier with an eluate. By this step, the cells and/or extracellular vesicles of interest adsorbed onto the solid-phase carrier are detached from the solid-phase carrier and transferred into the eluate.

In the first separation/purification method, the eluate is used for detaching the cells and/or extracellular vesicles of interest adsorbed onto the solid-phase carrier from the solid-phase carrier. As the eluate, for example, an aqueous solution having a salt concentration suitable for elution (for example, sodium chloride aqueous solution) can be used.

By this step, the cells and/or extracellular vesicles of interest detached from the solid-phase carrier are transferred into the eluate, but if necessary, the eluate containing the cells and/or extracellular vesicles of interest is collected. This operation enables the obtainment of a solution containing cells and/or extracellular vesicles and having a decreased amount of impurities as compared to the original liquid sample.

### Second Separation/Purification Method

The second method for separating/purifying cells and/or extracellular vesicles according to the present invention comprises: bringing a solution containing cells and/or extracellular vesicles of interest into contact with the solid-phase carrier of the present invention; and collecting cells and/or extracellular vesicles of interest not adsorbed onto the solid-phase carrier, with no limitation on other steps.

### (Contact Step)

The contact step in the second separation/purification method of the present invention is a step of bringing a solution containing cells and/or extracellular vesicles of interest (also referred to as a liquid sample) into contact with the solid-phase carrier of the present invention. In the second separation/purification method, by this step, impurities to be removed in the liquid sample are adsorbed onto the solid-phase carrier, and separated from the cells and/or extracellular vesicles of interest in the liquid sample.

In the present invention, for the liquid sample, an arbitrary solution containing cells and/or extracellular vesicles of interest can be used, and the description of the first separation/purification method can be applied.

In the present invention, the selectivity on cells and/or extracellular vesicles depends on an ion exchanger fixed on the solid-phase carrier. Thus, in the second separation/purification method, the solid-phase carrier is selected for use according to impurities to be removed in the liquid sample.

Here, the solution of the liquid sample is adjusted so that it has an osmotic pressure equal to that of body fluid, for example. In the case of such a liquid sample, for example, when the liquid sample and the solid-phase carrier of the present invention come into contact with each other, impurities to be removed are adsorbed onto the solid-phase carrier, and cells and/or extracellular vesicles are not adsorbed onto it.

When the liquid sample and the solid-phase carrier of the present invention come into contact with each other, for example, the pH of an environment surrounding an anion-exchange solid carrier is, for example, neutral or alkaline. Specifically, the pH may be, for example, 7 to 9.

### (Collection Step)

The collection step in the second separation/purification of the present invention is a step of collecting cells and/or extracellular vesicles of interest not adsorbed onto the solid-phase carrier. In this step, a solution containing cells and/or extracellular vesicles of interest not adsorbed onto the solid-phase carrier in the contact step are collected. This step enables the obtainment of a solution containing cells and/or extracellular vesicles and having a decreased amount of impurities as compared to the original liquid sample.

### Preferred Aspects

In the first and second separation/purification methods of the present invention, a solid-phase carrier suitable for separation/purification is used. For example, a solid-phase carrier with a substrate such as a device, a tool, a material or a reagent that is commonly used in the field of biological separation/purification methods can be preferably used. More specifically, a solid-phase carrier, in which a column filler is used as a substrate, and an ion exchanger is fixed on a surface of the substrate, can be preferably used. In addition, a solid-phase carrier, in which a plate for a separation/purification stack is used as a substrate, and an ion exchanger is fixed on a surface of the substrate, can be preferably used. In the first and second separation/purification methods of the present invention, more specifically, it is preferable to use a column for separating/purifying cells and/or extracellular vesicles, filled with the solid-phase carrier of the present invention, or a stack for separating/purifying cells and/or extracellular vesicles, comprising the solid-phase carriers of the present invention stacked as a stack plate.

### Method for Fixing Cells and/or Extracellular Vesicles Onto Solid-Phase

The method for fixing cells and/or extracellular vesicles onto a solid-phase carrier of the present invention comprises: adsorbing cells and/or extracellular vesicles onto the solid-phase carrier of the present invention, with no limitation of other steps.

### (Adsorption Step)

The adsorption step in the fixation method of the present invention is a step of adsorbing cells and/or extracellular vesicles onto the solid-phase carrier of the present invention. By this step, cells and/or extracellular vesicles to be detected can be adsorbed onto the solid-phase carrier, and thus fixed onto the solid phase. Here, for the adsorption step, the description of the above-described adsorption step in the detection method of the present invention can be applied. The fixation method of the present invention enables easy and convenient fixation without necessity to apply a centrifugal force using a centrifuge or the like as described for the above-described adsorption step.

### EXAMPLES

Hereinafter, the present invention will be described in detail by giving Examples, which are intended for better understanding of the present invention, and in no way intended to limit the scope of the present invention.

### Preparation of Ion Exchanger-Fixed Glass Slide

An ion exchanger is fixed on a glass slide by coating a surface of the glass slide with an amino group (using, for example, a silane coupling agent such as 3-aminopropyltriethoxysilane), and binding the ion exchanger to the amino group. Here, PEI or DEAE was used as the ion exchanger. PEI was fixed by binding PEI to the amino group on the glass slide using glutaraldehyde as a cross-linker. DEAE was fixed by binding 2-diethylaminoethylamine to the amino group on the glass slide using glutaraldehyde as a cross-linker. In the present Example, a slide glass coated with an amino group in advance was prepared instead of coating a surface of the slide glass with an amino group.

By the following procedure, the ion exchanger (PEI or DEAE) was fixed to the glass slide coated with an amino group (a high-water-repellent print glass slide, MAS-coated, 12 holes, TF1205M; manufactured by Matsunami Glass Ind., Ltd.). The surface roughness (Ra) of the glass slide was about 4 nm.

### (PEI-Fixed Glass Slide)

First, a 2% glutaraldehyde solution (cross-linker) was added in an amount of 30 μL per hole to the amino group-coated glass slide, and left standing at room temperature for 2 hours. Next, the glass slide was washed with ion-exchange water for removing an unreacted cross-linker and by-products. After the washing, a 1 to 3% PEI solution (branched type with a high molecular weight (molecular weight: 60,000 to 80,000); manufactured by nacalai tesque) was added in an amount of 30 μL per hole to the glass slide, and left standing at room temperature for 2 hours. Thereafter, the surface of the glass slide was washed with ion-exchange water to remove an unreacted ion exchanger and by-products, thereby preparing a PEI-fixed glass slide. Thereafter, a 0.1 mol/L borate buffer was added in an amount of 30 μL per hole, followed by storage until use.

### (DEAE-Fixed Glass Slide)

A 2% glutaraldehyde solution (cross-linker) was added in an amount of 30 μL per hole to the amino group-coated glass slide, and left standing at room temperature for 2 hours. Next, the glass slide was washed with ion-exchange water for removing an unreacted cross-linker and by-products. After the washing, a 1 to 2% 2-diethylaminoethylamine solution (manufactured by Tokyo Chemical Industry Co., Ltd.) was added in an amount of 30 μL per hole to the glass slide, and left standing at room temperature for 2 hours. Thereafter, the surface of the glass slide was washed with ion-exchange water to remove an unreacted ion exchanger and by-products, thereby preparing a DEAE-fixed glass slide. Thereafter, PBS was added in an amount of 30 μL per hole, followed by storage until use.

### Fixation (Adsorption) of Extracellular Vesicles Onto Ion Exchanger-Fixed Glass Slide

In the present Example, sample solutions containing extracellular vesicles (EVs) were prepared, which are as follows.
Human-derived extracellular vesicles: A human adipose tissue mesenchymal stem cell culture supernatant was partially purified by an ultrafiltration method (using a 100 kDa cut-off filter), and used as a human-derived extracellular vesicle sample.
Escherichia coli-derived extracellular vesicles: DH 5α strain-derived EVs (Cosmo Bio Company, Limited) was used as a sample.
Lactic acid bacteria-derived extracellular vesicles: A commercially available lactic acid bacteria beverage was partially purified by an ultrafiltration method (using a 100 kDa cut-off filter), and used as a lactic acid bacteria-derived extracellular vesicle sample.
Lemon-derived extracellular vesicle sample: 100% Lemon juice (POKKA LEON 100; POKKA SAPPORO Food & Beverage Ltd.) was partially purified by an ultrafiltration method (using a 100 kDa cut-off filter), and used as a lemon-derived extracellular vesicle sample.
Human urine sample: Urine was collected from a healthy person, and used as a human urine sample.
Human blood plasma sample: Blood plasma was collected from a healthy person, and used as a human blood plasma sample.

The extracellular vesicles were adsorbed and fixed onto the ion exchanger-fixed glass slide by the following procedure. First, the ion exchanger-fixed glass slide was washed with ion-exchange water, and then tapped to remove the buffer. Next, the sample solution containing EVs was added to the glass slide in an amount of 4 μL per hole, and left standing at 4° C. for 2 hours to adsorb and fix EVs in the sample solution onto the glass slide.

### Detection of Extracellular Vesicles

The EVs fixed on the glass slide was fluorescently stained, and observed with a fluorescent microscope to detect the EVs. In the present Example, the fluorescent staining was performed using the following reagents.
Anti-human CD9 antibody (mouse monoclonal (12A12); manufactured by Cosmo Bio Company, Limited)
Anti-human CD63 antibody (mouse monoclonal (8A12); manufactured by Cosmo Bio Company, Limited)
Alexa Fluor 647-labeled anti-human GPNMB antibody (mouse monoclonal (D-9); manufactured by Santa Cruz Biotechnology)
Rhodamine-labeled polymyxin B (rhodamine B-labeled polymyxin B; manufactured by Sigma-Aldrich Co. LLC)
FITC-labeled vancomycin (manufactured by Sigma-Aldrich Co. LLC).

The fluorescent staining of EVs was performed by the following procedure. First, EVs were fixed onto the glass slide as described above, the sample solution supernatant was then removed, and a blocking solution (0.2 M L-lysine, 2% BSA-PBS; or FBS) was added in an amount of 30 μL per hole, and left standing at 4° C. for 60 minutes for blocking. When the staining was performed using an antibody which had not been fluorescently labeled, the blocking solution was removed, and 4 μL of an antibody diluted to an appropriate concentration was then added, and left standing at 4° C. for 30 minutes. Thereafter, washing with PBS was performed, and 4 μL of a fluorescently labeled secondary antibody diluted to an appropriate concentration was then added, and left standing at 4° C. for 30 minutes. When direct staining with a fluorescently labeled stain (rhodamine-labeled polymyxin B or FITC-labeled vancomycin) or a fluorescently labeled antibody (Alexa Fluor 647-labeled anti-human GPNMB antibody) was performed, the blocking solution was removed, and 4 μL of a stain or an antibody diluted to an appropriate concentration was then added, and left standing at 4° C. for 30 minutes.

After the fluorescent staining, the reagent supernatant was removed, the glass slide was then washed with ion-exchange water, moisture was removed by suction, and fluorescently stained EVs on the glass slide was observed using a fluorescent microscope (manufactured by KEYENCE CORPORATION). In FIG. 4, a super-resolution microscope (HM-1000; manufactured by Sysmex Corporation) was used instead of the above-described fluorescent microscope (conventional fluorescent microscope).

### Results

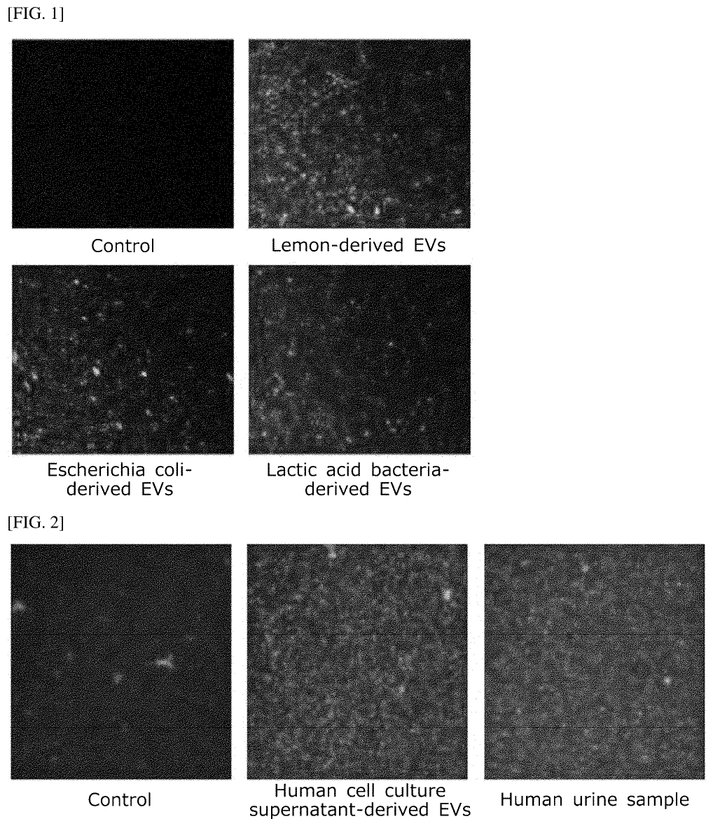

FIG. 1 shows the photographs of Escherichia coli-derived EVs, lactic acid bacteria-derived EVs or lemon-derived EVs which were adsorbed and fixed onto a PEI-fixed glass slide, stained with rhodamine-labeled polymyxin B, and then photographed using a fluorescent microscope. Here, the polymyxin B-reactive cell membrane is dyed red. For the control, 2% BSA (in PBS) was used as a sample solution.

From FIG. 1, it has been confirmed that various EVs can be adsorbed onto the PEI-fixed glass slide, and can be fluorescently stained, and detected with a fluorescent microscope.

FIG. 2 shows the photographs of human cell culture supernatant-derived EVs or EVs in a human urine sample which were adsorbed and fixed onto a DEAE-fixed glass slide, treated with an anti-human CD9 antibody and an anti-human CD63 antibody, then stained with a rhodamine-labeled anti-mouse IgG antibody, and then photographed using a fluorescent microscope. Here, the human-derived EVs are dyed red. For the control, 2% BSA (in PBS) was used as a sample solution.

From FIG. 2, it has been confirmed that human cell culture supernatant-derived EVs can be adsorbed and fixed onto the DEAE-fixed glass slide, and can be detected with a fluorescent microscope. Further, it has been confirmed that EVs in a human urine sample can also be adsorbed and fixed onto the DEAE-fixed glass slide, and can be fluorescently stained and detected with a fluorescent microscope.

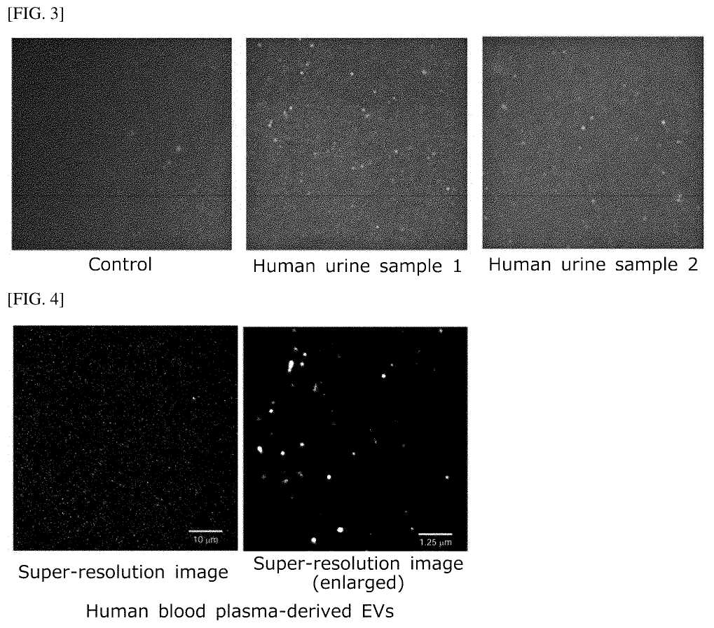

FIG. 3 shows the photographs of EVs in a human urine sample which are adsorbed and fixed onto a PEI-fixed glass slide, double-stained with rhodamine-labeled polymyxin B and FITC-labeled vancomycin, and then photographed using a fluorescent microscope. Here, the Gram-positive bacteria-derived EVs are dyed green with FITC-labeled vancomycin, and the Gram-negative bacteria-derived EVs are dyed red with rhodamine-labeled polymyxin B. For the control, 2% BSA (in PBS) was used as a sample solution. As the human urine samples, urine samples obtained from two healthy persons were used (human urine sample 1 and human urine sample 2).

From FIG. 3, it has been confirmed that Gram-positive bacteria-derived EVs and Gram-negative bacteria-derived EVs can be detected at the same time on the PEI-fixed glass slide.

Therefore, it has been found that by using a PEI-fixed glass slide, a plurality of types of EVs in a sample solution can be fixed onto one slide glass, and the EVs of different types can be separately detected.

FIG. 4 shows the photographs of EVs in a human blood plasma which was adsorbed and fixed onto a PEI-fixed glass slide, stained with Alexa Fluor 647-labeled anti-human GPNMB antibody, and then photographed using a super-resolution fluorescent microscope. Here, the human-derived EVs are dyed red.

From FIG. 4, it has been confirmed that human blood plasma-derived EVs can be adsorbed and fixed onto a PEI-fixed glass slide, and detected with a super-resolution microscope. Further, an image with an ordinary fluorescent microscope did not enable definite analysis of the size of particles fixed on the glass slide because the resolution was not sufficient, but analysis of a super-resolution image with a super-resolution microscope showed that the particles on the glass slide had a size of several tens to 200 nm which is expected for EVs.

This application claims priority based on Japanese patent application No. 2023-197552 filed on Nov. 21, 2023, the disclosure of which is incorporated herein in its entirety.

### INDUSTRIAL APPLIICABILITY

According to the present invention, a new technique for trapping extracellular vesicles (EVs) can be provided. The technique of the present invention, in which not an antibody but an ion exchanger is used for trapping EVs, thus is advantageous in terms of cost and ease of handling. In the technique of the present invention, cells can also be targeted for trapping. For this reason, the technique of the present invention is useful for development of EVs-related products.

1. A solid-phase carrier having an ability to adsorb cells and/or extracellular vesicles, the solid-phase carrier comprising a substrate and an ion exchanger.

2. The solid-phase carrier according to claim 1, wherein the ion exchanger and the substrate are bound to each other by a covalent bond, either via a connection part or without a connection part.

3. The solid-phase carrier according to claim 1, wherein the ion exchanger is a weak anion exchanger having a secondary and/or tertiary amino group as a partial structure.

4. The solid-phase carrier according to claim 3, wherein the ion exchanger is a monovalent residue or a polyvalent residue derived from polyethyleneimine.

5. The solid-phase carrier according to claim 4, wherein the polyethyleneimine is branched high-molecular-weight polyethyleneimine.

6. The solid-phase carrier according to claim 2, wherein the ion exchanger and the substrate are bound to each other via a connection part, and the connection part comprises a silicon-carbon bond.

7. The solid-phase carrier according to claim 1, wherein a material of the substrate is glass or metal oxide.

8. The solid-phase carrier according to claim 1, wherein the substrate is a glass slide or a microscope glass plate.

9. The solid-phase carrier according to claim 8, wherein a material of the glass slide or the microscope glass plate is glass having a transmittance of 30% or more at a wavelength of 300 nm.

10. The solid-phase carrier according to claim 8, wherein in the glass slide or the microscope glass plate, a surface roughness (Ra) of the glass before the ion exchanger is fixed thereon is 10 nm or less.

11. The solid-phase carrier according to claim 8, wherein a surface of the solid-phase carrier is two-dimensionally separated into a hydrophilic region and a water-repellent region.

12. The solid-phase carrier according to claim 1, wherein the substrate is a column filler.

13. The solid-phase carrier according to claim 1, wherein the substrate is a plate for a separation/purification stack.

14. A kit for detecting or quantitating cells and/or extracellular vesicles, comprising the solid-phase carrier according to claim 1, and a substance for detecting cells and/or extracellular vesicles.

15. The kit according to claim 14, for use in examination/diagnosis.

16. The kit according to claim 15, wherein a target for the examination/diagnosis is at least one selected from the group consisting of a bowel disease, a liver disease, a kidney disease, a neuropsychiatric disease, a metabolic disease, a respiratory disease, a cardiovascular disease, an age-related disease, and heavy alcohol intake.

17. A method for detecting cells and/or extracellular vesicles, comprising:
adsorbing cells and/or extracellular vesicles onto the solid-phase carrier according to claim 1; and
detecting the cells and/or extracellular vesicles adsorbed onto the solid-phase carrier.

18. The detection method according to claim 17, further comprising: labeling cells and/or extracellular vesicles with a substance for detecting cells and/or extracellular vesicles.

19. The detection method according to claim 18, wherein the substance for detecting cells and/or extracellular vesicles is at least one selected from the group consisting of an antibody, lectin, a membrane-reactive substance, and a nucleic acid-reactive substance.

20. The detection method according to claim 17, wherein cells and/or extracellular vesicles are detected by microscope observation.

21. The detection method according to claim 17, wherein cells and/or extracellular vesicles are detected by an immunoassay method.

22. The detection method according to claim 17, for use in examination/diagnosis.

23. The detection method according to claim 22, wherein a target for the examination/diagnosis is at least one selected from the group consisting of a bowel disease, a liver disease, a kidney disease, a neuropsychiatric disease, a metabolic disease, a respiratory disease, a cardiovascular disease, an age-related disease, and heavy alcohol intake.

24. A column for separating/purifying cells and/or extracellular vesicles, comprising a separation/purification column filled with the solid-phase carrier according to claim 12.

25. A stack for separating/purifying cells and/or extracellular vesicles, comprising the solid-phase carriers according to claim 13 stacked as a stack plate.

26. A method for separating/purifying cells and/or extracellular vesicles, comprising:
bringing a solution containing cells and/or extracellular vesicles of interest into contact with the solid-phase carrier according to claim 1; and
eluting cells and/or extracellular vesicles of interest adsorbed onto the solid-phase carrier with an eluate.

27. A pharmaceutical composition comprising cells and/or extracellular vesicles separated/purified by the separation/purification method according to claim 26.

28. A method for separating/purifying cells and/or extracellular vesicles, comprising:
bringing a solution containing cells and/or extracellular vesicles of interest into contact with the solid-phase carrier according to claim 1; and
collecting cells and/or extracellular vesicles of interest not adsorbed onto the solid-phase carrier.

29. A pharmaceutical composition comprising cells and/or extracellular vesicles separated/purified by the separation/purification method according to claim 28.

30. A method for fixing cells and/or extracellular vesicles onto a solid-phase carrier, comprising:
adsorbing cells and/or extracellular vesicles onto the solid-phase carrier according to claim 1.
